(19) United States
(12) Patent Application Publication
(10) Pub. No.: US 2023/0285390 A1
(43) Pub. Date: Sep. 14, 2023

(54) METHODS OF TREATING PULMONARY INJURY WITH CGRP INHIBITORS

(71) Applicant: Biohaven Pharmaceutical Holding Company Ltd., New Haven, CT (US)

(72) Inventors: St John Donnie McGrath, Southwick, MA (US); Vladimir CORIC, Madison, CT (US); Charles M. CONWAY, Cheshire, CT (US); Reese M. Caldwell, Cambridge, MA (US); Steven M. SCHNITTMAN, Woodbridge, CT (US)

(21) Appl. No.: 17/912,540

(22) PCT Filed: Mar. 22, 2021
(86) PCT No.: PCT/US2021/023388

§ 371 Date: Sep. 19, 2022

Related U.S. Application Data
(60) Provisional application No. 62/993,451, filed on Mar. 23, 2020.

(51) Int. Cl. A61K 31/496; A61K 9/00; A61P 31/14; A61P 11/00; A61P 29/00; A61K 45/06

(52) U.S. Cl. CPC A61K 31/496; A61K 9/0043; A61K 45/06; A61P 11/00; A61P 29/00; A61P 31/14

(57) ABSTRACT

Provided is a method for treating COVID-19 in a patient in need of such treatment, wherein the method includes administration to the patient of a therapeutically effective amount of CGRP inhibitor. Also provided is a pharmaceutical composition for treating COVID-19 in a patient in need of such treatment, wherein the pharmaceutical composition includes a therapeutically effective amount of CGRP inhibitor.

### CROSS-REFERENCE TO RELATED APPLICATIONS

This application claims priority to U.S. Provisional Application No. 62/993,451 filed Mar. 23, 2020 and all the benefits accruing therefrom under 35 U.S.C. § 119, the content of which is incorporated herein in its entirety by reference.

### BACKGROUND OF THE INVENTION

Respiratory tract disorders present widespread problems throughout the world. They fall into a number of major categories, including inflammatory conditions, infections, trauma, embolism, and inherited diseases. Infections caused by viruses are among the most abundant respiratory tract disorders.

Coronaviruses are a large family of viruses which may cause illness in animals or humans. In humans, several coronaviruses are known to cause respiratory infections ranging from the common cold to more severe diseases such as Middle East Respiratory Syndrome (MERS) and Severe Acute Respiratory Syndrome (SARS). Coronavirus disease 2019 (COVID-19) is a respiratory illness that can spread from person to person. The virus that causes COVID-19 is a novel coronavirus (referred to as “SARS-CoV-2”) that was first identified during an investigation into an outbreak in Wuhan, China.

COVID-19 is the infectious disease caused by the most recently discovered coronavirus. The disease quickly spread around the world infecting hundreds of thousands people and resulting in pandemic. COVID-19 spreads primarily through contact with an infected person when they cough or sneeze. It also spreads when a person touches a surface or object that has the virus on it, then touches their eyes, nose, or mouth. The disease causes respiratory illness with flu-like symptoms such as a cough and fever. Most people infected with the COVID-19 virus will experience mild to moderate respiratory illness and recover without requiring special treatment. However, older people, and those with underlying medical problems like cardiovascular disease, diabetes, chronic respiratory disease, and cancer are more likely to develop serious illness that may result in death.

Patients with serious cases of COVID-19 experience pulmonary (lung tissue) injury. A common contributor to the pulmonary injury in many of these disorders is related to the influx of inflammatory cells, such as neutrophils, macrophages, and eosinophils. Inflammatory cells release noxious enzymes that can damage tissue and trigger physiologic changes. Elastases are one category of noxious enzyme that inflammatory cells release. Elastase enzymes degrade elastic fibers (elastin) in the lung. The damage caused by elastase enzymes may cause the release of tissue kallikrein (TK) and may trigger a cascade that attracts additional inflammatory cells to the lung. This influx of additional inflammatory cells release more elastase enzymes, and a “vicious cycle” of lung tissue damage ensues. There are no therapies available today to halt the progression of COVID-19.

CGRP (calcitonin gene-related peptide) is a 37 amino acid neuropeptide, which belongs to a family of peptides that includes calcitonin, adrenomedullin and amylin. In humans, two forms of CGRP (a-CGRP and 13-CGRP) exist and have similar activities. They vary by three amino acids and exhibit differential distribution. At least two CGRP receptor subtypes may also account for differential activities. The CGRP receptor is located within pain-signaling pathways, intracranial arteries and mast cells and its activation is known to play a causal role in migraine pathophysiology.

CGRP is also known as a key neurotransmitter in the neuro-immune axis (Assas et al. “Calcitonin gene-related peptide is a key neurotransmitter in the neuro-immune axis” Frontiers in Neuroscience, 2014, 14, 23). CGRP neuropeptide is released by nociceptive (pain) neurons and multiple other cell types in response to variety of external (infection, chemical, thermal, mechanical) and internal stimuli, primarily via transient receptor potential (TRP) ion channel activation. CGRP released by activation of TRPs is a key neuropeptide involved in the interaction between the nervous and immune systems at barrier surfaces on the human body. CGRP release is known to mediate inflammation via swelling, increased blood flow, and edema. It increases IL-6 and other proinflammatory cytokines (IL-17, IL-9) and polarizes T-cell differentiation towards Tₕ2 and Tₕ17 (Kabata H. et al. “Neuro-immune Crosstalk and Allergic Inflammation” J. Clin. Invest. 2019, 130, 1475-1482).

Both positive-stranded (rhinovirus) and negative-stranded (RSV, measles) RNA viruses have been shown to upregulate TRP channels. Activation of upregulated TRPs is a putative cause for the cough reflex in respiratory infection, where increased TRP channels result in increased Ca²⁺ beneficial for viral replication. Diverse TRP activation converges to release of CGRP, which mediates edema and neurogenic inflammation (Benemei S. et al. “TRP Channels and Migraine: Recent Developments and New Therapeutic Opportunities” Pharmaceuticals, 2019, 12, 54).

Accordingly, new therapies for the treatment of COVID-19 are desired.

### SUMMARY OF THE INVENTION

By the present invention, it may be possible to treat COVID-19 by the administration of a CGRP inhibitor either alone or in combination with other therapeutically effective agents. Provided is a method for treating COVID-19 in a patient in need of such treatment, including administering to the patient a therapeutically effective amount of CGRP inhibitor.

Also provided is a method for reversing, alleviating, ameliorating, inhibiting, slowing down or preventing the onset, progression, development, severity or recurrence of a symptom, complication or condition, or biochemical indicia associated with COVID-19 in a patient, including administering to the patient a therapeutically effective amount of CGRP inhibitor.

Also provided is a method for preventing COVID-19 in a patient, including administering to the patient a therapeutically effective amount of CGRP inhibitor.

Also provided is a method for treating pulmonary edema associated with COVID-19 in a patient in need of such treatment, including administering to the patient a therapeutically effective amount of CGRP inhibitor.

Also provided is a method for treating neurogenic inflammation associated with COVID-19 in a patient in need of such treatment, including administering to the patient a therapeutically effective amount of CGRP inhibitor.

Also provided is a method for treating a disorder associated with COVID-19 characterized by upregulation of transient receptor potential channel, including administering to a patient in need of such treatment a therapeutically effective amount of CGRP inhibitor.

Also provided is a method for slowing down or preventing transmission of bacterial or viral infection associated with COVID-19 from a patient to another person, including administering to the patient a therapeutically effective amount of CGRP inhibitor.

A pulmonary injury suitable for treatment in accordance with the present invention is a viral lung injury caused by SARS-CoV-2. The pulmonary injury may be pulmonary inflammation, such as, for example, pulmonary inflammation associated with COVID-19, e.g., pneumonia.

The CGRP inhibitor may include a CGRP antibody, a CGRP receptor antibody, an antigen-binding fragment from a CGRP antibody or a CGRP receptor antibody, a CGRP infusion inhibitory protein, a CGRP bio-neutralizing agent, a CGRP receptor antagonist, a small molecule CGRP inhibitor, or a polypeptide CGRP inhibitor.

In an aspect, the CGRP inhibitor may include a CGRP antibody, a CGRP receptor antibody, or an antigen-binding fragment from a CGRP antibody or a CGRP receptor antibody. The antigen-binding fragment may include one or both of a heavy chain variable region and a light chain variable region from a CGRP antibody or a CGRP receptor antibody. The heavy chain variable region may include HCDR1, HCDR2, and HCDR3 from the heavy chain variable region of CGRP antibody or CGRP receptor antibody and/or wherein the light chain variable region comprises LCDR1, LCDR2, and LCDR3 from the light chain variable region of CGRP antibody or CGRP receptor antibody. The heavy chain variable region and/or the light chain variable region may include the heavy chain variable region and/or the light chain variable region of CGRP or CGRP receptor antibody. The CGRP antibody may be selected from galcanezumab-gnlm, fremanezumab-vfrm, eptinezumab-jjmr, and erenumab-aooe.

In another aspect, the CGRP inhibitor may be a small molecule CGRP inhibitor. The CGRP inhibitor may be a CGRP receptor antagonist. The CGRP receptor antagonist may be selected from olcegepant, telcagepant, ubrogepant, atogepant, rimegepant, and zavegepant. In an embodiment, the CGRP receptor antagonist may be rimegepant. In another embodiment, the CGRP receptor antagonist may be zavegepant. The CGRP inhibitor may be administered intranasally or nose-to-brain.

The method may further include administering an interleukin inhibitor to the patient. The interleukin inhibitor may be an IL-6 inhibitor, an IL-9 inhibitor, an IL-17 inhibitor, or a combination thereof. In an embodiment, the IL-6 inhibitor may be at least one selected from ACTEMRA° (tocilizumab) and SYLVANT° (siltuximab). For example, the IL-6 inhibitor may be ACTEMRA° (tocilizumab). In another embodiment, the IL-6 inhibitor may be at least one selected from olokizumab (CDP6038), elsilimomab, BMS-945429 (ALD518), sirukumab (CNTO136), levilimab (BCD-089), and CPSI-2364.

The IL-17 inhibitor may be at least one selected from COSENTYX® (secukinumab), TALTZ® (ixekizumab), and SILIQ® (brodalumab).

In yet another embodiment, the interleukin inhibitor may be at least one selected from ARCALYST® (rilonasept), ILARIS® (canakinumab), KINERET® (anakinra), CINQAIR® (reslizumab), STELARA® (ustekinumab), FACENRA® (benralizumab), NUCALA® (mepolizumab), DUPIXENT® (dupilumab), ILUMYA® (tildrakizumab), TREMFYA® (guselkumab), KEVZARA® (sarilumab), SIMULECT® (basiliximab), SKYRIZI® (risankizumab), ZENAPAX® (daclizumab), and ZINBRYTA® (daclizumab).

The method may further include administering an anti-viral agent to the patient. The anti-viral agent may include remdesivir, ritonavir, lopinavir, or a combination thereof. The anti-viral agent may further include interferon beta. In an embodiment, the anti-viral agent may include remdesivir. In another embodiment, the anti-viral agent may include ritonavir and lopinavir. The anti-viral agent may further include interferon beta.

The method may further include administering an anti-bacterial agent to the patient. The anti-bacterial agent may include an anti-malarial agent. In an embodiment, the anti-malarial agent may include chloroquine, hydroxychloroquine, azithromycin, or a combination thereof. In another embodiment, the anti-malarial agent may include hydroxychloroquine and azithromycin.

Also provided is a pharmaceutical composition comprising a CGRP inhibitor and at least one selected from an interleukin inhibitor, an anti-viral agent, and an anti-bacterial agent.

Also provided is a kit for treating a condition associated with COVID-19 in a patient. The kit may include a pharmaceutical composition and instructions for administering the pharmaceutical composition. The kit may further include an apparatus for administering the pharmaceutical composition, e. g., an inhaler or nebulizer.

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As used herein, the term “administering” refers to the physical introduction of a composition comprising a therapeutic agent to a subject, using any of the various methods and delivery systems known to those skilled in the art. Administering can also be performed, for example, once, a plurality of times, and/or over one or more extended periods and can be a therapeutically effective dose or a subtherapeutic dose.

As used herein, the term “antibody” (Ab) refers to, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. As used herein, the term “isotype” refers, without limitation, to the antibody class or subclass (e.g., IgM or IgG1) that is encoded by the heavy chain constant region genes. In certain embodiments, one or more amino acids of the isotype can be mutated to alter effector function. As used herein, the term “antibody” includes, by way of example, both naturally occurring and non-naturally occurring Abs; monoclonal and polyclonal Abs; chimeric and humanized Abs; human or nonhuman Abs; wholly synthetic Abs; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

As used herein, the terms “in combination with” and “in conjunction with” refer to administration of one treatment modality in addition to another treatment modality. As such, “in combination with” or “in conjunction with” refers to administration of one treatment modality before, during, or after administration of the other treatment modality to the subject.

The term “pharmaceutically acceptable salt” refers to a salt form of one or more of the compounds described herein which are typically presented to increase the solubility of the compound in the gastric or gastroenteric juices of the patient’s gastrointestinal tract in order to promote dissolution and the bioavailability of the compounds. Pharmaceutically acceptable salts include those derived from pharmaceutically acceptable inorganic or organic bases and acids, where applicable. Suitable salts include, for example, those derived from alkali metals such as potassium and sodium, alkaline earth metals such as calcium, magnesium and ammonium salts, among numerous other acids and bases well known in the pharmaceutical art.

The terms “subject” and “patient” refer any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

The terms “effective amount”, “therapeutically effective amount”, “therapeutically effective dosage” and “therapeutically effective dose” of an agent (also sometimes referred to herein as a “drug”) refers to any amount of the agent that, when used alone or in combination with another agent, protects a subject against the onset of a disease or promotes disease regression evidenced by a decrease in severity of disease symptoms, an increase in frequency and duration of disease symptom-free periods, or relief from impairment or disability due to the disease affliction. The therapeutically effective amount of an agent can be evaluated using a variety of methods known to the skilled practitioner, such as in human subjects during clinical trials, in animal model systems predictive of efficacy in humans, or by assaying the activity of the agent in in vitro assays.

The term “treatment” refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.

With respect to the disease, “treatment” is an approach for obtaining beneficial or desired clinical results. For purposes of this invention, beneficial or desired clinical results include, but are not limited to, one or more of the following: improvement in any aspect of a major symptom including lessening severity, alleviation of major symptom intensity, and other associated symptoms, reducing frequency of recurrence, increasing the quality of life of those suffering from the symptom, and decreasing dose of other medications required to treat the symptom.

The starting materials useful for making the pharmaceutical compositions of the present invention are readily commercially available or can be prepared by those skilled in the art.

Sensory neurotransmitters have been extensively studied and their ability to affect different body functions has been shown in a range of studies. One of the main sensory neurotransmitters involved in immune function is calcitonin gene-related peptide (CGRP). CGRP exemplifies a neuroimmune connector, since it is released at the site of stimulation, affecting immediate responses as well as mediating information flow to the rest of the nervous system. CGRP is a critical, highly expressed sensory signal, making it an important member of neuro-immune communication pathways. C fibers, the smallest diameter unmyelinated sensory neurons, are the main source of this neuropeptide. Their small diameter generates one of the lowest threshold response elements in the nervous system indicating their vital role. To date, this low threshold has placed them in the category of nociceptive neurons as they are the first to register damage/toxins through the pain pathway. This categorization is reinforced by the fact that c fibers express on their surface the transient receptor potential vanilloid 1 (TRPV1) which is a key responder to tissue damage. However, below the pain threshold, C fibers are likely to be playing a critical role in physiological systems and in particular, in host monitoring and activation of host defense and immune responses due to their low activation potential.

CGRP is released in response to activation of TRPV1 in both the nervous and immune systems. In the nervous system, TRPV1 is expressed along the entire length of the sensory c fiber neurons, from the periphery to the somata in the CNS. These neurons innervate every organ and tissue in the body. Although a key exogenous ligand for TRPV1 is capsaicin, TRPV1 is also activated by a range of other endogenous agonists including heat (>43° C.), protons(~pH 4.5), lipids like anandamide, phosphatidylinositol(4,5)-biphosphate (PIP2), and voltage (FIG. 1). Heat and low pH activate TRPV1 by distinct molecular recognition sites (Assas et al. “Calcitonin gene-related peptide is a key neurotransmitter in the neuro-immune axis” Frontiers in Neuroscience, 2014, 14, 23, and references cited therein).

Sensory neurons are heterogeneous with respect to their sensitivity to stimuli, conduction velocity (myelination), and neuropeptide content. Each sensory nerve terminal expresses various combinations of ion channels to sense a variety of stimulations, including Naᵥ1.7, Naᵥ1.8, Naᵥ1.9, transient receptor potential vanilloid 1 (TRPV1), transient receptor potential ankyrin 1 (TRPA1), and transient receptor potential cation channel subfamily M member 8 (TRPM8) (FIG. 2). TRPV1 is responsive to high temperature and capsaicin, whereas TRPA1 mainly responds to chemical and mechanical stress as well as chemical irritants, including wasabi, and cold temperature. TRPM8 is responsive to cold temperature and menthol. A specialized subset of sensory neurons detecting noxious or potentially harmful stimuli are called nociceptors, which innervate skin, joints, respiratory, and gastrointestinal tract. Most nociceptors are small-diameter, unmyelinated, slowly conducting nerves referred to as C-fibers. Nociceptors express not only TRPA1 and TRPV1 but also various receptors for cytokines, lipid mediators, and growth factors, including ATP, adenosine, 5-hydroxytryptamine, cysteinyl leukotrienes, and protease-activated receptors. Therefore, a variety of stimulants, including inflammatory mediators, leads to the activation of nociceptors through these receptors (FIG. 2). For example, type 2 cytokines, such as IL-4, IL-5, and IL-13, induce sensory nerve activation and induce chronic itch. In addition, thymic stromal lymphopoietin (TSLP) has recently been found to activate TRPA1 by binding to its receptor, TSLPR, on sensory nerves in the skin of atopic dermatitis patients. Furthermore, Th2 cell-derived IL-31 activates TRPV1+TRPA1+ sensory nerves and induces mast cell-independent itch. Notably, the terminals of nociceptors contain neuropeptides, such as CGRP, substance P, and VIP, which are rapidly released in response to noxious stimuli and inflammation. These neuropeptides directly act on various immune cells (FIG. 2). Substance P is known to be a proinflammatory neuropeptide that activates multiple immune cells, including T cells, macrophages, DCs, mast cells, eosinophils, and neutrophils. The functions of VIP and CGRP skew toward a Th2 cytokine-like phenotype. Moreover, VIP suppresses inflammatory cytokines derived from DCs and macrophages, whereas it promotes Th2 cell differentiation, survival, and migration, and CGRP induces mast cell degranulation and shifts Langerhans cells to promote Th2 differentiation. These neuropeptides also affect nonimmune cells and increase vascular permeability, which is involved in the further recruitment of immune cells (Kabata H. et al. “Neuro-immune Crosstalk and Allergic Inflammation” J. Clin. Invest. 2019, 130, 1475-1482 and references cited therein).

Transient receptor potential (TRP) channels are a family of cation channels expressed primarily on the cell membrane that cluster into six families including TRPA, TRPC, TRPM, TRPP, TRPL, and TRPV. These channels are likely to contribute to a number of different physiological processes ranging from thermosensation and pain to regulation of Ca²⁺ levels in the endoplasmic reticulum.

Multiple TRP channels are expressed on trigeminal sensory neurons innervating the meninges including TRPV1, TRPA1, TRPV4, and TRPM8. These channels respond to stimuli implicated in migraine, both from a pathology perspective (e.g., acrolein on TRPA1) and a therapeutic perspective (e.g., parthenolide on TRPA1). Additional modulators are listed below their respective TRP channels.

Activation of TRP channels on meningeal afferents leads to action potential signaling into the trigeminal nucleus caudalis (left) and ultimately to headache (FIG. 3). Activation of TRP channels on these neurons also leads to the release of neuropeptides such as CGRP, activating CGRP receptors on blood vessels (right and bottom), causing vasodilation and contributing to neurogenic inflammation. Although not shown, TRP channels are also expressed on the central terminals of meningeal afferents, and CGRP is released as a transmitter in this synapse, both of which may also contribute to signaling within this circuit. Multiple migraine therapeutics may act in this circuit, including: BoNTA, which may indirectly contribute to decreased CGRP release and possibly inhibit recruitment of TRP channels to the membrane; GEPANTs, which block the CGRP receptor; anti-CGRP mAbs, which sequester extracellular CGRP; and anti-CGRP-R mAbs, which bind to and block the CGRP receptor (Benemei S. et al. “TRP Channels and Migraine: Recent Developments and New Therapeutic Opportunities” Pharmaceuticals, 2019, 12, 54, and references cited therein).

There is evidence that acute lung injury (thermal, chemical, viral) leads to upregulation of TRP channels and then activation of CGRP. This results in both acute lung injury (pulmonary edema with acute phase cytokine/mediator release) followed by chronic lung injury with hyaline membrane formation, fibrosis and reduced diffusion capacity. Acute Respiratory Distress Syndrome (ARDS), which is a common pathway resulting from diverse types of lung injury is part of this pathogenic process. The immunologic milieu surrounding the alveoli makes a shift toward Tₕ17 cytokines, including IL-6 and IL-17, that appears to be common, regardless of inciting agent.

Studies show that a heavily-polarized Tₕl7 immune response is a hallmark of SARS-type lung damage. FIG. 4 illustrates that IL-17 is the most upregulated cytokine in MERS patients. FIG. 5 illustrates flow cytometry of COVID-19 patient T-cells shows Tₕ17 response. Given that Tₕ17 cells are pro-fibrotic in multiple organs, including the lung, preventing Tₕ17 polarization by inhibiting CGRP receptors may decrease fibrotic complications of COVID-19. Accordingly, CGRP inhibition may mitigate COVID-19 complications - both in acute inflammatory/viral replication stage (characterized by IL-6 elevation), and progressive ALI/ARDS stage (IL-17/Tₕ17 driven pulmonary changes).

COVID-19 infection goes through a similar pathologic progression with acute lung injury changes, which if not reversed by the human host immune system, may progress to chronic, irreversible lung damage. It is reasonable to expect that, at least in part, that TRP mediated upregulation of CGRP and consequent immunologic shift to Tₕ17 cytokines and mediators contributes to the pulmonary pathogenesis of COVID-19 resulting in pulmonary injury. This preliminary data may suggest that inhibition of CGRP can block the pulmonary inflammation that is secondary to chemical or other incitement.

In accordance with the present invention, a patient having pulmonary injury associated with COVID-19 may take a therapeutically effective amount of CGRP inhibitor. The pulmonary injury may, for example, be a viral lung injury caused by SARS-associated coronavirus.

The patient may also suffer from another pulmonary injury which may be associated with pulmonary inflammatory disorders, chronic cough, common cold, pandemic flu, pneumonia, acute respiratory distress syndrome, severe acute respiratory syndrome, middle east respiratory syndrome, croup, acute lung injury, idiopathic respiratory distress syndrome, or idiopathic pulmonary fibrosis pulmonary hypertension, neonatal pulmonary hypertension, neonatal bronchopulmonary dysplasia, pulmonary embolism, chronic obstructive pulmonary disease, acute bronchitis, chronic bronchitis, emphysema, bronchiolitis, bronchiectasis, radiation pneumonitis, hypersensitivity, pleural effusion, pertussis, pleurisy, pneumonitis, asbestosis, acute inflammatory asthma, acute smoke inhalation, allergic asthma, work-related asthma, iatrogenic asthma, tuberous sclerosis, cystic fibrosis, tuberculosis, lung cancer, sarcoidosis, sleep apnea, spirometry, sudden infant death syndrome, alveolar proteinosis, or alpha-L-protease deficiency. The pulmonary inflammation may be associated with two or more of the above disorders in addition to COVID-19.

The pulmonary injury may be treated by administering a CGRP inhibitor, which may include a CGRP antibody, a CGRP receptor antibody, an antigen-binding fragment from a CGRP antibody or a CGRP receptor antibody, a CGRP infusion inhibitory protein, a CGRP bio-neutralizing agent, a CGRP receptor antagonist, a small molecule CGRP inhibitor, or a polypeptide CGRP inhibitor. The antigen-binding fragment may include one or both of a heavy chain variable region and a light chain variable region from a CGRP antibody or a CGRP receptor antibody. The heavy chain variable region may include HCDR1, HCDR2, and HCDR3 from the heavy chain variable region of CGRP antibody or CGRP receptor antibody and/or wherein the light chain variable region comprises LCDR1, LCDR2, and LCDR3 from the light chain variable region of CGRP antibody or CGRP receptor antibody. The heavy chain variable region and/or the light chain variable region may include the heavy chain variable region and/or the light chain variable region of CGRP or CGRP receptor antibody.

Thus, in an aspect, the CGRP inhibitor may be a biologic, which may be selected from i.e., antibodies, antibody fragments or peptides. Such biologics comprise molecules that have a mass of greater than about 900 Daltons, for example, greater than 1,100 Daltons, greater than 1,300 Daltons, greater than 1,500 Daltons, greater than 5,000 Daltons, greater than 10,000 Daltons, greater than 50,000 Daltons, or greater than 100,000 Daltons. Examples of CGRP biologics commercially available or currently being studied include the following. EMGALITY™ (galcanezumab-gnlm), available from Eli Lilly and Company, is a humanized IgG4 monoclonal antibody specific for calcitonin-gene related peptide (CGRP) ligand. Galcanezumab-gnlm is produced in Chinese Hamster Ovary (CHO) cells by recombinant DNA technology. Galcanezumab-gnlm is composed of two identical immunoglobulin kappa light chains and two identical immunoglobulin gamma heavy chains and has an overall molecular weight of approximately 147 kDa. AJOVY™ (fremanezumab-vfrm) injection, available from Teva Pharmaceutical Industries, is a fully humanized IgG2Da/kappa monoclonal antibody specific for calcitonin gene-related peptide (CGRP) ligand. Fremanezumab-vfrm is produced by recombinant DNA technology in Chinese hamster ovary (CHO) cells. The antibody consists of 1324 amino acids and has a molecular weight of approximately 148 kDa. VYEPTI™ (eptinezumab-jjmr), available from H. Lundbeck A/S, is a fully humanized IgG1 antibody manufactured using yeast (Pichia pastoris). AIMOVIG™ (erenumab-aooe) injection, available from Amgen Inc., is a human immunoglobulin G2 (IgG2) monoclonal antibody that has high affinity binding to the calcitonin gene-related peptide receptor. Erenumab-aooe is produced using recombinant DNA technology in Chinese hamster ovary (CHO) cells. It is composed of 2 heavy chains, each containing 456 amino acids, and 2 light chains of the lambda subclass, each containing 216 amino acids, with an approximate molecular weight of 150 kDa.

In another aspect, the CGRP inhibitor may be a small molecule CGRP inhibitor. For example, the CGRP inhibitor may be a CGRP receptor antagonist, which may be selected from olcegepant, telcagepant, ubrogepant, atogepant, rimegepant, and zavegepant.

Rimegepant has the chemical formula, C₂₈H₂₈F₂N₆O₃ and the IUPAC name [(5S,6S,9R)-5-amino-6-(2,3-difluorophenyl)-6,7,8,9-tetrahydro-5H-cyclohepta[b]pyridin-9-yl] 4-(2-oxo-3H-imidazo[4,5-b]pyridin-1-yl)piperidine-1-carboxylate. Rimegepant is also known as and referred to herein as BHV-3000.

The structure of rimegepant is:

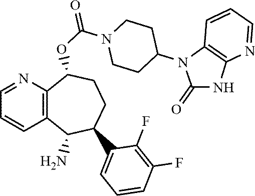 [SMILES: N[C@@H]1c2cccnc2[C@H](OC(=O)N2CCC(n3c(=O)[nH]c4ncccc43)CC2)CC[C@H]1c1cccc(F)c1F]

Rimegepant is described, for example, in WO 2011/046997 published Apr. 21, 2011.

In a preferred aspect of the invention, rimegepant may be present in the form of a hemisulfate sesquihydrate salt. This preferred salt form is described in WO 2013/130402 published Sep. 6, 2013.

The chemical formula of the salt form is C₂₈H₂₈F₂N₆O₃ • 0.5 H₂SO₄ • 1.5 H₂O and the structure is as follows:

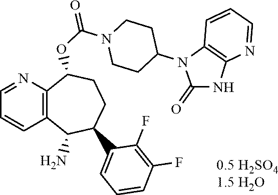

Another CGRP antagonist is zavegepant (previously known as “vazegepant”), which is described in WO 2011/123232 published Oct. 6, 2011, and has the following structure (also known as BHV-3500):

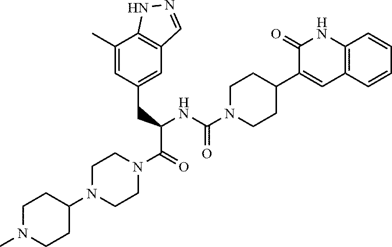 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

Another CGRP antagonist is ubrogepant, which has the following structure:

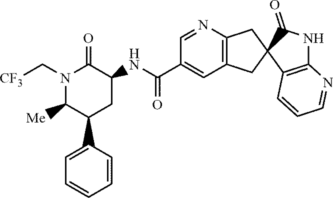 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

Another CGRP antagonist is atogepant, which has the following structure:

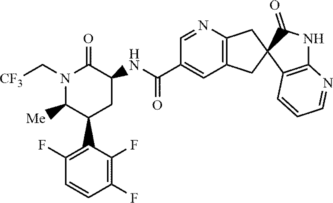 [SMILES: C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

Another CGRP antagonist is olcegepant, which has the following structure:

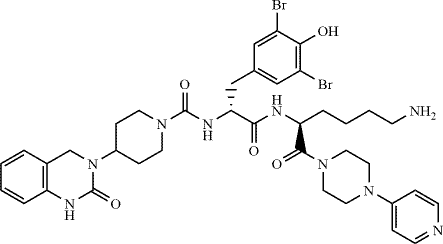 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

Typically, in accordance with the present invention, the CGRP inhibitor taken to treat pulmonary injury is administered in the form of a pharmaceutical composition, which may be prepared in any suitable dosage form including, for example, such as tablets, capsules, powders, granules, ointments, solutions, suppositories, injections, inhalants, gels, microspheres, and aerosols.

The pharmaceutical compositions of the present invention comprising a CGRP inhibitor typically also include other pharmaceutically acceptable carriers and/or excipients such as, for example, binders, lubricants, diluents, coatings, disintegrants, barrier layer components, glidants, coloring agents, solubility enhancers, gelling agents, fillers, proteins, co-factors, emulsifiers, solubilizing agents, suspending agents, flavorants, preservatives and mixtures thereof. A skilled artisan in the art would know what other pharmaceutically acceptable carriers and/or excipients could be included in the formulations according to the invention. The choice of excipients would depend on the characteristics of the compositions and on the nature of other pharmacologically active compounds in the formulation. Appropriate excipients are known to those skilled in the art (see Handbook of Pharmaceutical Excipients, fifth edition, 2005 edited by Rowe et al., McGraw Hill) and have been utilized to yield a novel sublingual formulation with unexpected properties.

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose, polyvinylpyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinyl pyrrolidone, agar, or alginic acid or a salt thereof such as sodium alginate. In an aspect of the invention, the flavoring agent is selected from mint, peppermint, berries, cherries, menthol and sodium chloride flavoring agents, and combinations thereof. In an aspect of the invention, the sweetener is selected from sugar, sucralose, aspartame, acesulfame, neotame, and combinations thereof.

In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes and the like.

In an aspect, the CGRP inhibitor is administered at a dose of about 1-1000 mg per day. In another aspect, the CGRP inhibitor is administered at a dose of about 1, 5, 10, 15, 20, 25, 30, 40, 50, 60, 70, 80, 90, 100, 200, 250, 300, 400, 500, 750, or 1000 mg per day. In an aspect, the CGRP inhibitor may be administered orally. In another aspect, the CGRP inhibitor may be administered intranasally or nose-to-brain. An example of an orally administered CGRP inhibitor is rimegepant. An example of intranasally or nose-to-brain administered CGRP inhibitor is zavegepant.

Other typical routes of administering the pharmaceutical compositions of the invention include, without limitation, topical, transdermal, inhalation, parenteral, sublingual, buccal, rectal, and vaginal. The term “parenteral” as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000).

Solid compositions are normally formulated in dosage units providing from about 1 to about 1000 mg of the active ingredient per dose. Some examples of solid dosage units are 0.1 mg, 1 mg, 10 mg, 37.5 mg, 75 mg, 100 mg, 150 mg, 300 mg, 500 mg, 600 mg and 1000 mg. Typical dose ranges in accordance with the present invention include from about 10-600 mg, 25-300 mg, 25-150 mg, 50-100 mg, 60-90 mg, and 70-80 mg. Liquid compositions are generally in a unit dosage range of 1-100 mg/mL. Some examples of liquid dosage units are 0.1 mg/mL, 1 mg/mL, 10 mg/mL, 25 mg/mL, 50 mg/mL, and 100 mg/mL.

In an aspect, the pharmaceutical composition may include about 50-60 weight% rimegepant hemisulfate sesquihydrate, about 30-35 weight% microcrystalline cellulose, about 2-7 weight% hydroxypropyl cellulose, about 3-7 weight% croscarmellose sodium, and about 0.1-1.0 weight% magnesium stearate. In another aspect, the pharmaceutical composition may include about 57.1 weight% rimegepant hemisulfate sesquihydrate, about 33.4 weight% microcrystalline cellulose, about 4.0 weight% hydroxypropyl cellulose, about 5.0 weight% croscarmellose sodium, and about 0.5 weight% magnesium stearate. In another aspect, the pharmaceutical composition may include from about 70-80 weight% rimegepant hemisulfate sesquihydrate, about 10-20 weight% fish gelatin, about 10-20 weight% of a filler, and 0.1-5.0 weight% of a flavorant.

Medical devices known to those skilled in the art such as inhalers and nebulizers may be used to administer the CGRP inhibitors to a patient in accordance with the present invention. Such devices include, for example, metered dose inhalers, dry powdered inhalers, soft mist inhalers and nebulizers. Such devices are readily commercially available.

The method, in accordance with the present invention, may further include administering an interleukin inhibitor to the patient, either independently, or in combination with the CGRP inhibitor. The interleukin inhibitor may be an IL-6 inhibitor, an IL-9 inhibitor, an IL-17 inhibitor, or a combination thereof. In an embodiment, the CGRP inhibitor may be administered in combination with ACTEMRA® (tocilizumab), an IL-6 receptor antagonist available from Genentech USA, Inc. In another embodiment, the CGRP inhibitor may be administered in combination with SYLVANT® (siltuximab), an IL-6 inhibitor available from Janssen Biotech, Inc. Examples of other IL-6 inhibitors which may be used in combination with the CGRP inhibitor are olokizumab (CDP6038), elsilimomab, BMS-945429 (ALD518), sirukumab (CNTO136), levilimab (BCD-089), and CPSI-2364. Examples of IL-17 inhibitors include COSENTYX® (secukinumab) available from Novartis International AG, TALTZ® (ixekizumab) available from Eli Lilly and Company, and SILIQ® (brodalumab) available from Bausch Health Companies, Inc. Examples of other interleukin inhibitors which may be used in combination with the CGRP inhibitor may include ARCALYST® (rilonasept), ILARIS® (canakinumab), KINERET® (anakinra), CINQAIR® (reslizumab), STELARA® (ustekinumab), FACENRA® (benralizumab), NUCALA® (mepolizumab), DUPIXENT® (dupilumab), ILUMYA® (tildrakizumab), TREMFYA® (guselkumab), KEVZARA® (sarilumab), SIMULECT® (basiliximab), SKYRIZI® (risankizumab), ZENAPAX® (daclizumab), and ZINBRYTA® (daclizumab).

In accordance with the present invention, the CGRP inhibitor may be administered in combination with an anti-viral medicine or anti-infective medicine. For example, the CGRP inhibitor may be administered in combination with remdesivir (GS-5734) developed by Gilead Sciences, Inc., NORVIR® (ritonavir) available from AbbVie, Inc., lopinavir, or KALETRA® (a combination of ritonavir and lopinavir) available from AbbVie, Inc. The combination may further include interferon beta. In an embodiment, rimegepant may be administered in combination with remdesivir. In another embodiment, rimegepant may be administered in combination with KALETRA®, and optionally, interferon beta.

In another example, the CGRP inhibitor may be administered with an anti-bacterial agent, for example, anti-malarial agent. The anti-bacterial agent may include chloroquine (CQ), hydroxychloroquine (HCQ), azithromycin, or a combination thereof. In an embodiment, rimegepant may be administered with chloroquine (CQ), hydroxychloroquine (HCQ), azithromycin, or a combination of chloroquine (CQ) or hydroxychloroquine (HCQ) and azithromycin.

In an aspect, the invention also provides kits for use in the instant methods. Kits can include one or more containers comprising a pharmaceutical composition described herein and instructions for use in accordance with any of the methods described herein. Generally, these instructions comprise a description of administration of the pharmaceutical composition to treat, ameliorate or prevent pulmonary injury, according to any of the methods described herein. The kit may, for example, comprise a description of selecting an individual suitable for treatment based on identifying whether that individual has pulmonary injury or whether the individual is at risk of having pulmonary injury. The instructions are typically provided in the form of a package insert, or label, in accordance with the requirements of the regulatory having authority over the jurisdiction where the pharmaceutical composition is to be provided to patients.

In another embodiment, a method for treating pulmonary edema associated with COVID-19 in a patient in need of such treatment may include administering to the patient a therapeutically effective amount of CGRP inhibitor.

In another embodiment, a method for treating neurogenic inflammation associated with COVID-19 in a patient in need of such treatment may include administering to the patient a therapeutically effective amount of CGRP inhibitor.

In another embodiment, method for reversing, alleviating, ameliorating, inhibiting, slowing down or preventing the onset, progression, development, severity or recurrence of a symptom, complication or condition, or biochemical indicia associated with pulmonary injury associated with COVID-19 in a patient may include administering to the patient a therapeutically effective amount of CGRP inhibitor.

In another embodiment, a method for preventing pulmonary injury associated with COVID-19 in a patient may include administering to the patient a therapeutically effective amount of CGRP inhibitor.

In another embodiment, a method for treating pulmonary edema associated with COVID-19 in a patient in need of such treatment may include administering to the patient a therapeutically effective amount of CGRP inhibitor.

In another embodiment, a method for treating neurogenic inflammation associated with COVID-19 in a patient in need of such treatment may include administering to the patient a therapeutically effective amount of CGRP inhibitor.

In another embodiment, a method for treating a disorder characterized by upregulation of transient receptor potential channel associated with COVID-19 comprising administering to a patient in need of such treatment a therapeutically effective amount of CGRP inhibitor.

In another embodiment, a method for slowing down or preventing transmission of bacterial or viral infection associated with COVID-19 from a patient to another person may include administering to the patient a therapeutically effective amount of CGRP inhibitor.

The description of all of these methods is the same or similar to the description provided above for the method of treating pulmonary injury associated with COVID-19 by administering a therapeutically effective amount of CGRP inhibitor.

The following example is provided for illustrative purposes and is not intended to limit the scope of the claims which follow.

### Example 1- Treatment of COVID-19

The following protocol describes a clinical study for treating patients in accordance with the present invention.

| Brief Title: | Safety and Efficacy Trial of Zavegepant* Intranasal for Hospitalized Patients With COVID-19 Requiring Supplemental Oxygen. |
|---|---|
| Official Title: | BHV-3500-203: Double-Blind, Randomized, Placebo Controlled, Safety and Efficacy Trial of Zavegepant* (BHV-3500) Intranasal (IN) for Hospitalized Patients With COVID-19 Requiring Supplemental Oxygen. |
| | * BHV-3500, formerly “vazegepant”, is now referred to as “zavegepant” (za ve′ je pant). The World Health Organization (WHO) International Nonproprietary Names (INN) Expert Committee revised the name to “zavegepant” which was accepted by the United States Adopted Names (USAN ) Council for use in the U.S. and is pending formal adoption by the INN for international use. |
| Brief Summary: | The purpose of this study is to determine if a CGRP receptor antagonist may potentially blunt the severe inflammatory response at the alveolar level, delaying or reversing the path towards oxygen desaturation, ARDS, requirement for supplemental oxygenation, artificial ventilation or deathin patients with COVID-19 on supplemental oxygen. |
| Rationale: | Zavegepant is a potent CGRP receptor antagonist. Acute lung injury induces upregulation of TRP channels which activates CGRP leading to both acute lung injury (pulmonary edema with acute phase cytokine/mediator release, with immunologic milieu shift toward TH17 cytokines) followed by chronic lung injury with hyaline membrane formation, fibrosis and reduced diffusion capacity. ARDS, which is a common pathway resulting from diverse types of lung injury is part of this pathogenic process. Because COVID-19 (SARS2) infection leads to an acute insult of pulmonary epithelia, we postulate that a CGRP receptor antagonist may potentially blunt the severe inflammatory response at the alveolar level, delaying or reversing the path towards oxygen desaturation, ARDS, requirement for supplemental oxygenation, artificial ventilation or death. |
| | The data from this study will allow characterization of the relative safety and efficacy of intranasal (IN) of zavegepant versus placebo in the treatment of COVID-19 infection leading to hospitalization. |
| Study Type: | Interventional |
| Study Phase: | Phase 2, Phase 3 |
| Study Design: | Allocation: Randomized |
| | Intervention Model: Parallel Assignment |
| | Masking: Quadruple (Participant, Care Provider, Investigator, Outcomes Assessor) |
| | Primary Purpose: Treatment |
| Condition: | COVID-19 infection |
| Intervention: | Drug: Zavegepant (BHV-3500) -- 10 mg intranasal (IN) for 14 days |
| | Drug: Placebo -- Placebo Q8h for 14 days |
| Study Arms | Experimental: Zavegepant |
| | Zavegepant (BHV-3500) 10 mg intranasal (IN) Q8h for 14 days |
| | Intervention: Drug: Zavegepant (BHV-3500) |
| | Placebo Comparator: Placebo |
| | Placebo Q8h for 14 days |
| | Intervention: Drug: Placebo |
| Estimated Enrollment: | 120 subjects |
| Inclusion Criteria | 1. Subjects must provide informed consent in accordance with requirements of the study center’s institutional review board (IRB) or ethics committee prior to the initiation of any protocol-required procedures. |
| | 2. Subjects must agree to provide all requested demographic information (i.e., gender, race). |
| | 3. Subjects must have symptoms that require hospitalization with supplemental oxygen and / or non-invasive ventilation as determined by the admitting physician. The maximum nasal cannula O₂ concentration should be determined by the treating clinician and the limitations of the specific equipment. |
| | 4. Subjects must have symptoms that require hospitalization with supplemental oxygen and / or non-invasive ventilation as determined by the admitting physician. The maximum nasal cannula O₂ concentration should be determined by the treating clinician and the limitations of the specific equipment. |
| | 5. Concomitant investigational agents for the treatment of COVID-19 shall be permitted, but not required. |
| | 6. Ability to provide informed consent signed by study patient or legally acceptable representative. |
| | 7. Willingness and ability to comply with study-related procedures/assessments. |
| Exclusion Criteria | 1. Subjects in immediate need of invasive mechanical ventilation or extracorporeal membrane oxygenation (ECMO). |
| | 2. Subjects with an eGFR < 30 mL/min, at the Screening Visit. |
| | 3. Prisoners or subjects who are involuntarily incarcerated. |
| | 4. Subjects who are participating in any other investigational clinical trial while participating in this clinical trial. |
| | 5. Subjects who are under the age of 18 years. |
| | 6. Subjects who are pregnant (all potential female enrollees need to have a negative pregnancy test prior to IP administration). |
| | 7. Subjects with multi-organ failure. |
| | 8. Subjects who have received more than 48 hours of supplemental oxygen prior to randomization. |
| | 9. Subjects with prior significant pulmonary disease (e.g., severe COPD/ILD/CHF/IPF) are excluded. |
| | 10. Subjects receiving investigational therapies as part of a formal clinical trial for the treatment of COVID-19. During the course of this study, investigational therapies that may become “standard of care” to treat COVID-19, but are not part of a clinical trial, are allowed. |
| | 11. Subjects who are on long-acting CGRP monoclonal antibodies will be excluded including Aimovig® (erenumab), Emgality® (galcanezumab), Ajovy® (fremanezumab), and Vyepti® (eptinezumab). Additionally, the investigational oral CGRP receptor antagonist, atogepant, that is taken daily will also be excluded. Oral CGRP receptor antagonists, Nurtec ODT® (rimegepant) and Ubrelvy® (ubrogepant) that are typically used PRN infrequently will not be excluded as long the subject was not taking them on a daily basis and does not take them during the current study. |
| | 12. Subjects who are unlikely to survive for more than 48 hours from the Screening Visit. |
| | 13. Subjects with any of the following abnormal laboratory values at screening: aspartate AST or ALT greater than 5x ULN or bilirubin greater than 2x ULN. |
| | 14. Subjects with known active TB, history of incompletely treated TB, suspected or known extrapulmonary TB. |
| | 15. Subjects with suspected or known systemic bacterial or fungal infections. However, empiric antibiotics are permitted. |
| | 16. Subjects who have participated in any clinical research study evaluating an IP or therapy within 3 months and less than 5 half-lives of IP prior to the screening visit. |
| | 17. Subjects with any physical examination findings and/or history of any illness that, in the opinion of the study investigator, might confound the results of the study or pose an additional risk to the subject by their participation in the study. |
| Sex/Gender | Sexes Eligible for Study: All |
| Ages | 18 Years and older (Adult, Older Adult) |
| Accept Healthy Volunteers | No |
| Primary Outcome Measures: | To evaluate the safety and efficacy of zavegepant compared with placebo in patients hospitalized with COVID-19 infection requiring supplemental oxygen (time frame: Baseline to Day 15) |
| | 1 death |
| | 2 hospitalized, on invasive mechanical ventilation or ECMO |
| | 3 hospitalized, on non-invasive ventilation or high flow oxygen devices |
| | 4 hospitalized, requiring supplemental oxygen |
| | 5 hospitalized, not requiring supplemental oxygen |
| | 6 not hospitalized |
| Secondary Outcome Measures: | 1. Proportion of subjects who have a 6-point severity rating of 5 or 6, are alive, and do not use supplemental oxygen as a procedure at Day 29. [Time Frame: Baseline to Day 29]. |
| | 2. Proportion of subjects who have a 6-point severity rating of 2 or 3, or use any ventilation or high-flow nasal cannula as procedures, on any day through Day 29. [Time Frame: Baseline to Day 29]. |
| | 3. Proportion of subjects admitted into an ICU on any day through Day 29 from AE eCRFs. [Time Frame: Baseline to Day 29]. |
| | 4. Proportion of subjects who have a 6-point severity rating of 3, 4, 5, or 6, are alive, and do not use invasive mechanical ventilation as a procedure at Day 15. The analogous definition is applied to Day 29. [Time Frame: Baseline at Day 15 and at Day 29]. |
| | 5. Proportion of subjects who have a 6-point severity rating of 4, 5 or 6, or use a low- or high-flow nasal, are alive, and do not use any ventilation at Day 15. The analogous definition is applied to Day 29. [Time Frame: Baseline at Day 15 and at Day 29]. |
| | 6. Difference between treatment groups in the mean 6-point severity rating at Day 29. [Time Frame: Baseline to Day 29]. |
| | 7. Number of days from baseline to the first day through Day 29 with any 6-point severity rating greater than baseline. [Time Frame: Baseline to Day 29]. |
| | 8. Number of days from baseline to the first of any 2 consecutive days through Day 29 with all SpO2/FiO2 ratios > 400 on both days. [Time Frame: Baseline to Day 29]. |
| | 9. Number of days from baseline to the first day through Day 29 with ≥ 1-point decrease in any NEWS2 score from baseline. [Time Frame: Baseline to Day 29]. |
| | 10. Number of days from baseline to the first day through Day 29 with all NEWS2 scores < 2 on that day. [Time Frame: Baseline to Day 29]. |
| | 11. Mean change from baseline in NEWS2 score at Days 15 and 29 for subjects who are alive at these time points. [Time Frame: Baseline at Day 15 and at Day 29]. |
| | 12. Proportion of subjects who have a 6-point severity rating of 5 or 6, are alive, and do not use supplemental oxygen as a procedure at Day 15. [Time Frame: Baseline to Day 15]. |
| | 13. Proportion of subjects who are discharged from the hospital, have a 6-point severity rating of 6 on any day after discharge, and use supplemental oxygen on any day after discharge. [Time Frame: Baseline to Day 60]. |
| | 14. Mean number of days with respiratory rate > 24 breaths/minute through Day 29 for subjects who are alive at Day 29 and do not use invasive mechanical ventilation. [Time Frame: Baseline to Day 29]. |
| | 15. Mean number of days with supplemental oxygen use through Day 29 for subjects who are alive at Day 29. A day in which any 6-point severity rating is 2, 3, or 4, or supplemental oxygen is used as a procedure counts. [Time Frame: Baseline to Day 29]. |
| | 16. Number of days from baseline to the first day through Day 29 on which any SpO2 ≥ 90%, any 6-point severity rating is 5 or 6, and no supplemental oxygen is used as a procedure. [Time Frame: Baseline to Day 29]. |
| | 17. Mean number of ventilator-free days through Day 29 for subjects who are alive at Day 29. [Time Frame: Baseline to Day 29]. |
| | 18. Mean SOFA total scores at ICU admission and Day 29 (if still in ICU), from SOFA and AE eCRFs. [Time Frame: Baseline to Day 29]. |
| | 19. Mean number of days of hospitalization through Day 29 for subjects who are alive on Day 29. All days on study on or before hospitalization discharge are days of hospitalization, from 6-point severity rating scale eCRFs. [Time Frame: Baseline to Day 29]. |
| | 20. Number of days from baseline to the first of any 2 consecutive days through Day 29 in which all temperatures show lack of fever on both days and no antipyretics are used on either day. [Time Frame: Baseline to Day 29]. |
| | 21. Number of subjects with deaths, SAEs, severe AEs, and Grade 3 or 4 laboratory test abnormalities at any time on study. [Time Frame: Screening to Day 60]. |
| | 22. Number and percentage of subjects with severe or life-threatening bacterial, invasive fungal, or opportunistic infections at any time through Day 29 from AE/SAE eCRFs. [Time Frame: Baseline to Day 29]. |
| | 23. Number and percentage of subjects with intranasal administration reactions at any time through Day 29 from AE/SAE eCRFs. [Time Frame: Baseline to Day 29]. |
| | 24. Proportion of subjects with ≥ 50% reduction in eGFR from baseline at any time on study from laboratory test eCRFs. [Time Frame: Baseline to Day 60]. |
| Experimental Outcomes: | 1. CGRP levels |
| | 2. IL-6 levels |
| | 3. Procalcitonin levels |
| | 4. Others |

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A method for treating COVID-19 in a patient in need of such treatment, comprising administering to the patient a therapeutically effective amount of CGRP inhibitor.

2. The method of claim 1, wherein the treating the COVID-19 comprises reversing, alleviating, ameliorating, inhibiting, slowing down or preventing the onset, progression, development, severity or recurrence of a symptom, complication or condition, or biochemical indicia associated with pulmonary injury associated with COVID-19 in a the patient.

3. The method of claim 1, wherein the treating the COVID-19 comprises preventing pulmonary injury associated with COVID-19 in the patient.

4. The method of claim 1, wherein the treating the COVID-19 comprises treating pulmonary edema or neurogenic inflammation associated with COVID-19 in the patient in need of such treatment.

5. (canceled)

6. The method of claim 1, wherein the treating the COVID-19 comprises treating a disorder characterized by upregulation of transient receptor potential channel associated with COVID-19.

7. The method of claim 1, wherein the treating the COVID-19 comprises slowing down or preventing transmission of bacterial or viral infection from the patient to another person associated with COVID-19.

8. The method according to claim 1, wherein the patient has another pulmonary injury.

9. The method according to claim 8, wherein the other pulmonary injury is caused by influenza virus, parainfluenza virus, respiratory syncytial virus, human metapneumoviruses, adenovirus, rhinovirus, enterovirus, hantavirus, coronavirus, or a combination thereof.

10-12. (canceled)

13. The method according to claim 1, wherein the CGRP inhibitor comprises a CGRP antibody, a CGRP receptor antibody, an antigen-binding fragment from a CGRP antibody or a CGRP receptor antibody, a CGRP infusion inhibitory protein, a CGRP bio-neutralizing agent, a CGRP receptor antagonist, a small molecule CGRP inhibitor, or a polypeptide CGRP inhibitor.

14. The method according to claim 13, wherein the antigen-binding fragment comprises one or both of a heavy chain variable region and a light chain variable region from a CGRP antibody or a CGRP receptor antibody.

15. (canceled)

16. (canceled)

17. The method according to claim 13, wherein the CGRP antibody is selected from galcanezumab-gnlm, fremanezumab-vfrm, eptinezumab jjmr, and erenumab-aooe.

18. The method according to claim 13, wherein the CGRP receptor antagonist is selected from olcegepant, telcagepant, ubrogepant, atogepant, rimegepant, and zavegepant.

19. The method according to claim 18, wherein the CGRP receptor antagonist is rimegepant.

20. The method according to claim 18, wherein the CGRP receptor antagonist is zavegepant.

21. The method according to claim 7, wherein the CGRP inhibitor is administered intranasally or nose-to-brain or by delivery directly to the lungs of the patient.

22. The method according to claim 1 , further comprising administering an interleukin inhibitor to the patient.

23-28. (canceled)

29. The method according to claim 1, further comprising administering an anti-viral agent to the patient.

30-33. (canceled)

34. (canceled)

35. The method according to claim 1, further comprising administering an anti-bacterial agent to the patient.

36-38. (canceled)

39. A pharmaceutical composition comprising a CGRP inhibitor and at least one selected from an interleukin inhibitor, an anti-viral agent, or an anti-bacterial agent.

40. A kit for treating a condition associated with pulmonary injury in a patient, the kit comprising:
(a) the pharmaceutical composition of claim 39; and
(b) instructions for administering the pharmaceutical composition.

41. (canceled)
